(19) United States
(12) Patent Application Publication
(10) Pub. No.: US 2010/0324420 A1
(43) Pub. Date: Dec. 23, 2010

(54) Method and System for Imaging

(75) Inventors: Allen David Snook, Snohomish, WA (US); Matthew Bruce, Aix en Provence (FR); Rohit Garg, Kirkland, WA (US); Michael R. Vion, Lynnwood, WA (US); Dan M. Skyba, Snohomish, WA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 12/747,970

(22) PCT Filed: Dec. 8, 2008
(86) PCT No.: PCT/IB08/55148

§ 371 Date: Aug. 23, 2010

Related U.S. Application Data
(60) Provisional application No. 61/013,671, filed on Dec. 14, 2007.

(51) Int. Cl. A61B 8/14

(52) U.S. Cl. 600/443

(57) ABSTRACT

A method and system of imaging is provided. The system can include an imaging system (10) having at least one probe (120) for transmitting imaging energy into a region (150) of a body (50) and receiving response energy; a display device (170); and a processor (100) operably coupled to the at least one probe and the display device. The processor can generate a first image based on the response energy. The processor can present the first image on the display device. The processor can present on the display device a second image of the same plane or volume as the first image. The first image is different from the second image. The processor can retrieve a designation of a region of interest from a clinician that is associated with one of the first and second images. The processor can compare the first image with the second image for graphical differences, and the processor can present the region of interest on the other of the second image based at least in part on the graphical differences. Other embodiments are disclosed.

This disclosure relates generally to diagnostic systems and more specifically to a method and system for imaging.

Various forms of medical imaging can be used to non-invasively produce images of internal aspects of the body, including tissue, organs, muscles, tendons, vessels, blood flow, pathological lesions, and so forth. For example, a clinician can choose to utilize ultrasound imaging, x-ray imaging, computed tomography (CT), magnetic resonance imaging (MRI), fluoroscopy, nuclear medicine, positron emission tomography (PET), projection radiography, or photoacoustic imaging. The determination of which type of imaging to utilize is often based on what the clinician is looking for and where he or she is looking for it. For example, strain in a tissue or bloodflow through a vessel can be examined utilizing ultrasound imaging.

Ultrasonic imaging systems are capable of imaging and measuring the physiology within the body by transmitting ultrasonic waves into the body from the surface of the skin and reflecting the waves from physiology within the body, such as tissue and cells. The reflected echoes are received by an ultrasonic transducer and processed to produce an image of the plane or volume scanned by the beams.

The images or data can then be examined by a clinician as part of a diagnosis. To facilitate this process, ultrasonic contrast agents can be introduced into the body to enhance ultrasonic diagnosis. Contrast agents are substances which strongly interact with ultrasonic waves, returning echoes which may be clearly distinguished from those returned by blood and tissue. An example of an ultrasonic contrast agent utilized is microbubbles. Microbubbles can provide an acoustic impedance mismatch in the body, and nonlinear behavior in certain acoustic fields which is readily detectable through ultrasonic processing. Gases that have been stabilized in solutions in the form of tiny microbubbles can be infused into the body and survive passage through the pulmonary system and circulate throughout the vascular system. Microbubble contrast agents are useful for imaging the body's vascular system, for instance, as the contrast agent can be injected into the bloodstream and will pass through the veins and arteries of the body with the blood supply until filtered from the blood stream in the lungs, kidneys and liver.

However, image processing can often result in certain physiology of the body being clearer than other physiology. For instance, an image processing technique can clearly present the contrast agent in an image stream so that the clinician can analyze blood flow through a vessel or the heart, but that same image stream may not clearly depict structure, such as tissue or an organ, in proximity to the vessel within the same plane or volume being scanned. A clinician may be utilizing the ultrasound imaging to examine a particular area of the body without knowing whether it is the vessel or the surrounding structure that needs to be examined.

Use of separate ultrasound examinations for viewing different physiology of the body within a plane or volume of the body can be time consuming and requires locating the same place or volume. A region of interest selected by a clinician during one ultrasound examination may be different from a region of interest selected in a different ultrasound examination. This can complicate the review of the images and the diagnosis. Additionally, other modalities of imaging may be better suited for presenting images of other structure or other data within a particular plane or volume of the body.

Accordingly, there is a need for a method and system of imaging that facilitates review of the images or data by a clinician. There is a further need for a method and system of imaging that provides clearer images of a number of aspects of the body within the plane or volume being examined. There is yet a further need for a method and system of imaging that accurately presents a region of interest on the images.

The Summary is provided to comply with 37 C.F.R. §1.73, requiring a summary of the invention briefly indicating the nature and substance of the invention. It is submitted with the understanding that it will not be used to interpret or limit the scope or meaning of the claims.

In one exemplary embodiment of the present disclosure, a method of imaging is provided. The method can include transmitting ultrasonic waves into a region of a body and receiving echoes in response; generating at least two images based on the echoes, where the at least two images are different from each other; presenting the at least two images on a display device; retrieving a designation of a region of interest from a clinician that is associated with a first of the at least two images; comparing the first of the at least two images with another of the at least two images for graphical differences; and presenting the region of interest on the another of the at least two images based at least in part on the graphical differences.

In another exemplary embodiment, a computer-readable storage medium in which computer-executable code is stored, where the computer-executable code is configured to cause a computing device in which the computer-readable storage medium is loaded to execute a number of steps, is provided. The steps can include transmitting imaging energy into a region of a body and receiving response energy; generating a first image based on the response energy; generating a second image of the same plane or volume as the first image, where the first image is different from the second image; presenting the first and second images simultaneously on a display device; retrieving a designation of a region of interest from a clinician that is associated with one of the first and second images; comparing the first image with the second image for graphical differences; and presenting the region of interest on the other of the first and second images based at least in part on the graphical differences.

In a further exemplary embodiment, an imaging system is provided that can have at least one probe for transmitting imaging energy into a region of a body and receiving response energy; a display device; and a processor operably coupled to the at least one probe and the display device. The processor can generate a first image based on the response energy. The processor can present the first image on the display device. The processor can present on the display device a second image of the same plane or volume as the first image. The first image is different from the second image. The processor can retrieve a designation of a region of interest from a clinician that is associated with one of the first and second images. The processor can compare the first image with the second image for graphical differences, and the processor can present the region of interest on the other of the second image based at least in part on the graphical differences.

The technical effect includes, but is not limited to, facilitating the review by a clinician of images and data captured during an imaging examination. The technical effect further includes, but is not limited to, accurately translating a region of interest from one image to another image.

The above-described and other features and advantages of the present disclosure will be appreciated and understood by those skilled in the art from the following detailed description, drawings, and appended claims.

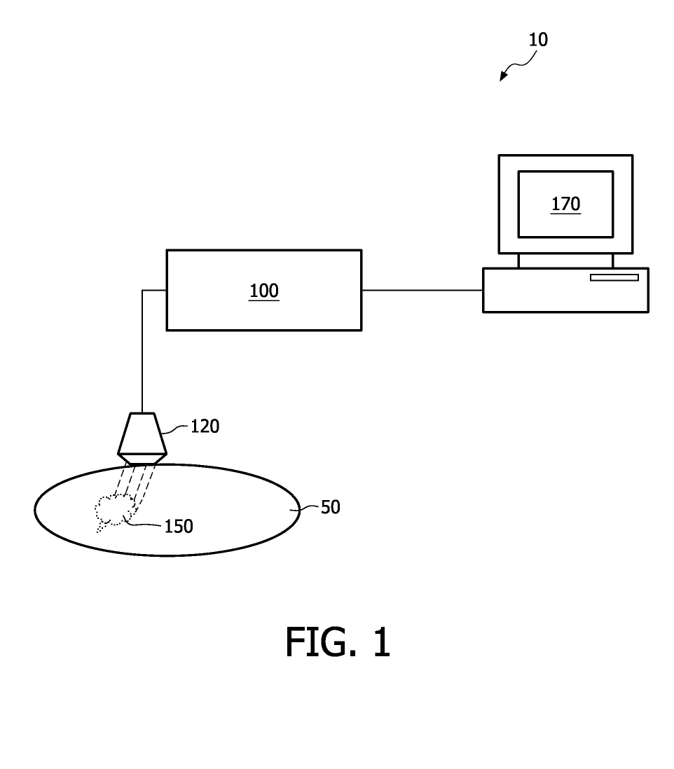

FIG. 1 is a schematic illustration of a system for performing imaging according to an exemplary embodiment of the present invention;

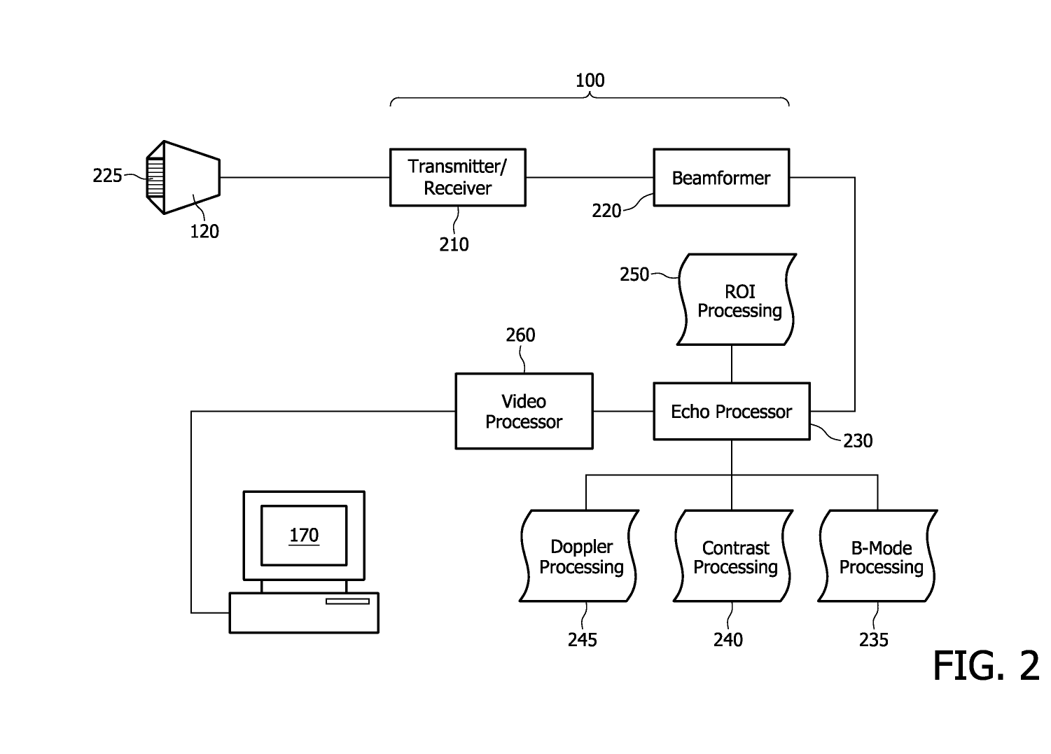

FIG. 2 is a schematic illustration of an exemplary embodiment of a processor that can be used with the system of FIG. 1;

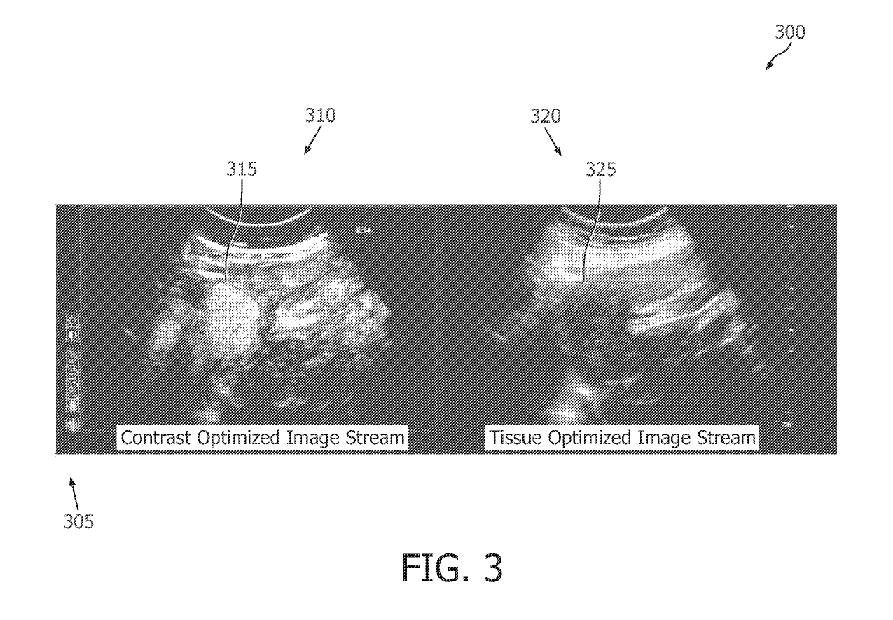

FIG. 3 is an image of an exemplary embodiment of a display presented by the system of FIG. 1;

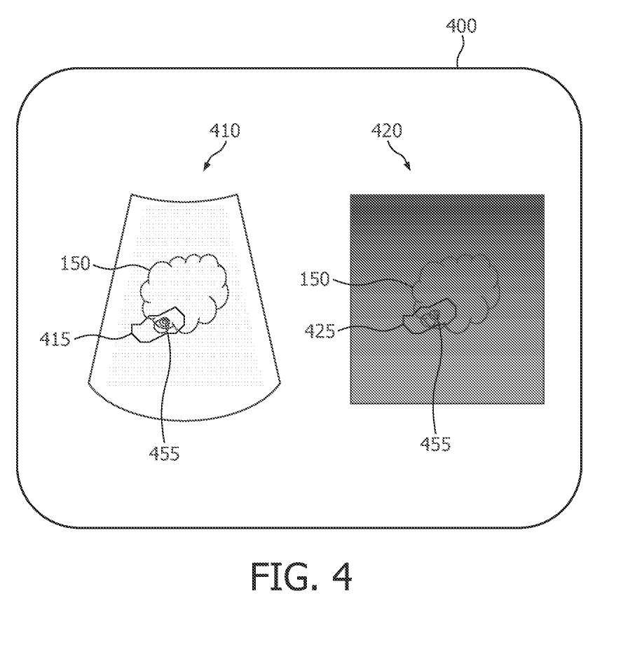

FIG. 4 is an image of another exemplary embodiment of a display presented by the system of FIG. 1;

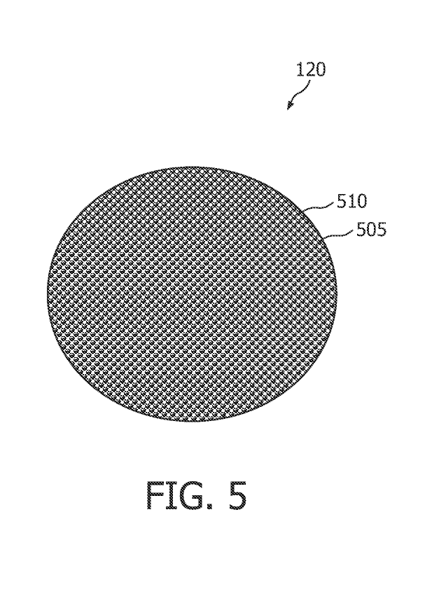

FIG. 5 is a schematic illustration of an exemplary embodiment of a transducer array for an ultrasonic probe that can be used with the system of FIG. 1; and

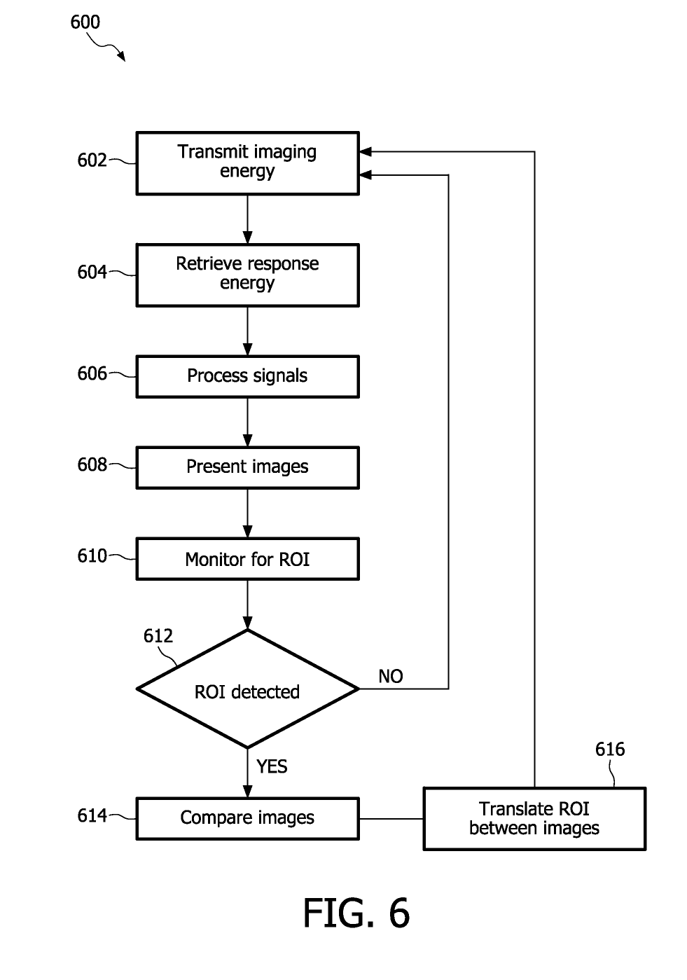

FIG. 6 is a method that can be used by the system of FIG. 1 for performing imaging according to an exemplary embodiment of the present invention.

The exemplary embodiments of the present disclosure are described with respect to data capture and imaging of a body performed by an ultrasound imaging device using a contrast agent. It should be understood by one of ordinary skill in the art that the exemplary embodiments of the present disclosure can be applied to various portions of the body, whether human or animal. The method and system of the exemplary embodiments of the present disclosure can also be used with other imaging systems, such as computed tomography (CT), magnetic resonance imaging (MRI), and with combinations of modalities of imaging, such as presenting images based upon data retrieved by an ultrasound examination and based upon data retrieved by a MRI examination.

Referring to the drawings, and in particular to FIG. 1, an ultrasound imaging system in accordance with one exemplary embodiment of the invention is shown and generally represented by reference numeral 10. The system 10 can perform ultrasound imaging on a patient's body 50, such as of an organ or tissue 150, and can include a processor or other control device 100, a probe or transducer 120, and a display device 170.

Referring additionally to FIG. 2, the processor 100 can include various components for performing ultrasound imaging, and can employ various imaging techniques, such as with respect to data capture, analysis and presentation. For example, the processor 100 can include a transmitter/receiver 210, a beamformer 220, an echo processor 230, and a video processor 260. The present disclosure also contemplates one or more of these components being combined. The ultrasonic probe 120 can include an array of ultrasonic transducer elements 225 which transmit and receive the ultrasonic energy, such as under the control of the beamformer 220. In one embodiment, the beamformer 220 can control the timing of actuation of the transducer array elements 225, such as formed into a linear array transducer, by activating transducer pulsers of the transmitter/receiver 210 at appropriate times. In another embodiment, the probe 120 can be a matrix array transducer that provides a steered and focused ultrasonic beam.

During reception, ultrasonic echoes received by the transducer elements 225 can be provided to the transmitter/receiver 210 which is coupled to the beamformer 220, where the signals are appropriately delayed then combined to form a sequence of coherent echo signals over the depth of reception in the body 50 of the patient. The beamformer 200 can provide the echo signals to the echo processor 230. In one embodiment, echoes received by the transducer probe 120 can be digitized by analog to digital converters (not shown).

The echo processor 230 can perform a number of signal processing techniques based upon a number of factors, such as the physiology of the body 50 that is to be imaged, the type of examination, the type of signal and so forth. For example, the echo processor 230 can have a B-Mode processing tool 235 or other software that processes the echo signals for presentation of a B-Mode image. The echo processor 230 can have a contrast processing tool 240 or other software that processes the signals to enhance imaging of the contrast agent. The echo processor 230 can have a Doppler processing tool 245 or other software that processes the signals to retrieve data such as power, flow and velocity characteristics.

The display device 170, with the assistance of the video processor 260, can then be used to present the image generated from the B-Mode processing, the image generated from the contrast tool processing, and/or other data retrieved by the Doppler processing. In one embodiment, two or more of these images can be presented on display device 170 simultaneously. Each of the different images presented can also be streams of images. In one embodiment, one or more of the streams of images can be real-time imaging. The present disclosure also contemplates one or more of the image streams being generated from previously collected data. For example, data collected can be used for generating the image streams which are then presented on the display device 170 as a continuous loop. The present disclosure also contemplates presenting a combination of real-time and looped image streams at the same time on the display device.

As described above, the exemplary embodiment of system 10 shows the use of ultrasound imaging to present two image streams where one image stream is a B-Mode scan and the other image stream is a contrast enhanced scan. However, the present disclosure also contemplates the use of other types of ultrasound images or image streams being simultaneously presented on display device 170 in addition to or in place of one or more of the B-mode and contrast enhanced image streams. For example, ultrasound elastograms depicting an image representative of the strain on a tissue can be presented by system 10 on the display device 170 while simultaneously presenting the B-Mode imaging stream as described above. In another embodiment, the B-Mode and/or contrast enhanced ultrasound imaging can be presented by system 10 on the display device 170 while simultaneously presenting images of the same or of a similar plane or volume from a different imaging modality, such as an MRI or CT image.

Referring additionally to FIG. 3, a display 300 of the display device 170 is shown. The display 300 can include a first image stream 310 (only one frame of which is shown) and a second image stream 320 (only one frame of which is shown). The first image stream 310 can be generated by system 10 using the contrast processing tool 240, while the second image stream 320 can be generated using a signal processing tool or software (not shown) that enhances tissue imaging. Display 300 can include a graphical user interface (GUI) tool 305 that allows a clinician to perform certain tasks with respect to the image streams 310, 320. In one embodiment, using the GUI tool 305, a clinician can designate a Region of Interest (ROI) 315 in the image stream 310. In another embodiment, a type of ROI 315 can be selected from a menu and a mouse cursor can be used to draw the ROI on the image. This can be done using GUI tool 305 or another GUI. System 10 using a ROI processing tool 250 or other software can then translate the location of ROI 315 onto the image stream 320 resulting in ROI 325.

The ROI processing tool 250 can determine differences between image streams, such as differences in apex, dimensions and/or scaling. The tool 250 can then compensate for those difference between the image streams when translating the position of the designated ROI on the first image stream to the same position on a second image stream. The tool 250 can determine the graphical data comprising the ROI (e.g., vertices, lines, splines, and annotations) and scale it for the image stream to which it is to be applied. The tool 250 can make the translation determinations thr positioning of the ROI on any number of other image streams, including image streams of a different modality.

It should be understood by one of ordinary skill in the art that ROI 320 can first be designated by the clinician which results in generation of ROI 325 in image stream 310. Additionally, where more than two images or image streams are presented on the display device 170, the present disclosure contemplates system 10 translating the ROI 315 or 325 onto one or more of the other image streams, such as according to a designation by the Clinician. It should further be understood by one of ordinary skill in the art that the ROI can be any type of designation or highlighting provided by the clinician, including fixed shapes, slices, volumes, and so forth. The exemplary embodiment describes GUI tool 305 as being utilized by the clinician for designating one of the ROIs 315, 325. However, the present disclosure also contemplates other tools and techniques for designating the ROI, including a touch-sensitive screen of display device 170 and a pointer for drawing the ROI on the screen.

Referring to FIG. 4, another exemplary embodiment of a presentation of image streams by system 10 on a display 400 of the display device 170 is shown. Display 400 can present a first image stream 410 (only one frame of which is shown) of the organ or tissue 150 and a second image 420 of the organ or tissue 150. The first image stream 410 can be generated by system 10 using the B-Mode processing tool 235, while the second image 420 can be generated using data retrieved from an examination performed using a different modality of imaging, such as an MRI exam. In this exemplary embodiment, the clinician can designate a ROI 415 in the image stream 410 where there appears to be a lesion 455 or some other area of interest. The ROI 415 can then be translated by ROI processing tool 250 from the same location with respect to the organ 150 onto the MRI image 420, resulting in presentation of the ROI 425.

In one embodiment, the data from the other imaging modality (e.g., the MRI exam) can be processed by system 10 to generate the image 420. In another embodiment, the data is processed by another device or system, such as a processor (not shown) associated with the MRI examination, and provided to the system 10 for presentation on display device 10. System 10 can have memory for storage of the data or images from the other imaging modalities and/or can be in communication with a database or the other imaging systems for retrieval of the data or images associated with the other imaging modality.

Referring back to FIG. 2, the system 10 is described above as generating two or more image streams (e.g., B-Mode and contrast enhanced image streams) from a single type of ultrasonic energy, such as pulsing ultrasonic waves from the transducer elements 225 having the same frequency and amplitude. However, the present disclosure also contemplates utilizing different ultrasonic energy for generating the two or more imaging streams to be presented on the display device 170. For example, pulses of ultrasonic enemy having a high amplitude and low frequency can be used for generating the B-Mode images, while pulses of ultrasonic energy having a low amplitude and high frequency can be used for generating the contrast enhanced images. In one embodiment, the probe 120 can transmit the different ultrasonic waves in an alternating fashion, although other pulsing patterns can be utilized. The particular pattern utilized for pulsing the ultrasonic energy can be chosen based on a number of factors, including the interval between pulses and the frequency and/or amplitude of the ultrasonic waves being pulsed.

Referring additionally to FIG. 5, the probe 120 can provide for different ultrasonic energy being transmitted through use of a first group of transducer elements 505 and a second group of transducer elements 510 that are co-located on the probe 120. The first and second groups of transducer elements 505, 510 can be arranged in an alternating pattern, although other positions of the first and second groups is also contemplated. The number and configuration of the first and second groups of transducer elements 505, 510 can be determined by the system 10 based on a number of factors, including the type of examination (e.g., contrast enhanced), the physiology being examined (e.g., the type of tissue) and/or the frequency and/or amplitude of the ultrasonic energy being transmitted. The probe 120 of FIG. 5 can transmit the different ultrasonic energy simultaneously, although the present disclosure contemplates other timing being applied to the pulses.

Referring additionally to FIG. 6, an exemplary method of operation of the system 10 is shown and generally represented by reference numeral 600. It would be apparent to an artisan with ordinary skill in the art that other embodiments not depicted in FIG. 6 are possible without departing from the scope of the claims described below, including examination of other portions of the body.

Method 600 can begin with step 602 in which the probe 120 transmits the imaging energy or waves, such as the ultrasonic pulses of system 10. The present disclosure also contemplates that the method 600 can be applied to other imaging techniques, such as transmitting radio waves into the magnetic field applied to a patient during an MRI exam or transmitting x-ray energy during a CT exam. In step 604, the system 10 retrieves the responsive energy, such as the ultrasound echoes. System 10 can then process the echoes in step 606 to enhance imaging as described above, such as for enhancing contrast and enhancing tissue images. Other image enhancements can also be applied by system 10, such as enhancing for different types of contrast agents or for different types of tissue.

In step 608, the images or image streams generated from the data processing of system 10 can be presented on the display device 170. The system 10 can monitor for selection or designation of an ROI, such as by the clinician, in step 610. If no ROI is designated in step 612 then the method 600 can repeat the previous steps, such as in real-time imaging, or revert back to the image loop while monitoring for an ROI designation. If an ROI has been designated on one of the image streams presented back in step 608 then the ROI processing tool 250 can compare the image streams for graphical differences, such as in the apex, dimensions or scaling, as in step 614. In step 616, the ROI can be translated from the location on the first imaging stream to the same location on the second imaging stream based on the comparative data retrieved in step 614.

The present disclosure also contemplates presenting two or more image streams that are of different portions of the same plane or volume, or of different planes or volumes. The system 10, based upon information regarding the spatial relationship between the different portions of the same plane or volume, or of the different planes or volumes, can then translate the ROI from one image or image stream to another image or image stream.

In one embodiment, the system 10 can also include a memory device, such as a CINELOOP® memory. Other components and/or techniques can also be used with the processor 100, such as an automatic border detection processor that can define and graphically overlay anatomical borders with respect to the images presented. The present disclosure also contemplates the use of other components and/or techniques in addition to, or in place of, the components of processor 100 described above.

According to another embodiment, the array of transducer elements 225 of the probe 120 can be a two dimensional array such as disclosed in U.S. Pat. No. 6,428,477, assigned to the assignee of the present disclosure and incorporated herein by reference. U.S. Pat. No. 6,428,477 discloses delivery of therapeutic ultrasound and performing ultrasound diagnostic imaging with the use of a two dimensional ultrasound array. The two dimensional ultrasound array includes a matrix or grid of transducer elements that allows three-dimensional (3D) images to be acquired, although 2D imaging is also contemplated by the present disclosure. The matrix of transducer elements makes possible the steering and electronic focusing of ultrasound energy in any arbitrary direction.

In one embodiment, the beamformer signals can be stored in an image data buffer (not shown) of the system 10, which stores image data for different volume segments of an image volume and for different points of a cardiac cycle. The image data can be output from the image data buffer to the display device 170, which generates a three-dimensional image of the region of interest from the image data. The display device 170 may include a scan converter which converts sector scan signals from the beamformer 220 to conventional raster scan display signals. Processor 100 can provide overall control of the ultrasound diagnostic imaging system, including timing and control functions.

In another embodiment, synthetic focus can be utilized by system 10 where each transducer element 225 or subset of transducer elements is actuated sequentially. The transmission from each element or group of elements can cover the entire image region. The echoes from each transmission can be received by all of the transducer elements 225 concurrently and stored. These echoes can then be combined in different combinations with different effective delays to form coherent echoes at points in the image region which are effectively focused at all points.

The invention, including the steps of the methodologies described above, can be realized in hardware, software, or a combination of hardware and software. The invention can be realized in a centralized fashion in one computer system, or in a distributed fashion where different elements are spread across several interconnected computer systems. Any kind of computer system or other apparatus adapted for carrying out the methods described herein is suited. A typical combination of hardware and software can be a general purpose computer system with a computer program that, when being loaded and executed, controls the computer system such that it carries out the methods described herein.

The invention, including the steps of the methodologies described above, can be embedded in a computer program product. The computer program product can comprise a computer-readable storage medium in which is embedded a computer program comprising computer-executable code for directing a computing device or computer-based system to perform the various procedures, processes and methods described herein. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. Therefore, it is intended that the disclosure not be limited to the particular embodiment(s) disclosed as the best mode contemplated for carrying out this invention, but that the invention will include all embodiments falling within the scope of the appended claims.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims.

1. A method of ultrasound imaging, the method comprising:
transmuting ultrasonic waves into a region of a body and receiving echoes in response;
generating at least two images based on the echoes, the at least two images being different from each other;
presenting the at least two images on a display device;
retrieving a designation of a region of interest from a clinician that is associated with a first of the at least two images;
comparing the first of the at least two images with another of the at least two images for graphical differences; and
presenting the region of interest on the another of the at least two images based at least in part on the graphical differences.

2. The method of claim 1, wherein the graphical differences comprise at least one of an apex, a dimension and a scaling.

3. The method of claim 1, further comprising transmitting at least two different types of ultrasonic waves to generate the echoes, the different types of ultrasonic waves having at least one of a different frequency and a different amplitude.

4. The method of claim 3, wherein the different types of ultrasonic waves are transmitted during different pulses.

5. The method of claim 3, wherein the different types of ultrasonic waves are transmitted simultaneously from a transducer array.

6. The method of claim 1, wherein the at least two images are presented on the display device simultaneously.

7. The method of claim 1, wherein one or more of the at least two images are presented on the display device in real-time.

8. The method of claim 1, wherein one of the at least two images is contrast enhanced or tissue enhanced.

9. The method of claim 1, further comprising electronically steering the ultrasonic waves into the region of the body.

10. The method of claim 1, wherein generating further comprises generating images of the same plane or volume.

11. The method of claim 10, wherein the images are generated by different imaging modalities.

12. An imaging system (10) comprising:
at least one probe (120) for transmitting imaging energy into a region (150) of a body (50) and receiving response energy;
a display device (170); and
a processor (100) operably coupled to the at least one probe and the display device, wherein the processor generates a first image based on the response energy, wherein the processor presents the first image on the display device, wherein the processor presents on the display device a second image of the same plane or volume as the first image, wherein the first image is different from the second image, wherein the processor retrieves a designation of a region of interest from a clinician that is associated with one of the first and second images, wherein the processor compares the first image with the second image for graphical differences, and wherein the processor presents the region of interest on the other of the second image based at least in part on the graphical differences.

13. The system (10) of claim 12, wherein the presenting of the first and second images is simultaneously on the display device (170).

14. The system (10) of claim 13, wherein the at least one probe (120) is an ultrasonic probe that transmits ultrasonic waves to generate the response energy, Wherein at least one of the first and second images is an image stream, and wherein the second image is generated based on the response energy.

15. The system (10) of claim 14, wherein the ultrasonic waves are pulses, and wherein a first of the pulses has at least one of a different frequency and a different amplitude from a second of the pulses.
